(19) United States
(12) Patent Application Publication
(10) Pub. No.: US 2024/0148638 A1
(43) Pub. Date: May 9, 2024

(54) Omni Hair Moisturizer

(71) Applicant: Abigail Aviv, Orlando, FL (US)

(72) Inventor: Abigail Aviv, Orlando, FL (US)

(21) Appl. No.: 18/052,554

(22) Filed: Nov. 3, 2022

(51) Int. Cl. A61K 8/9789; A61K 8/67; A61Q 5/12

(52) U.S. Cl. CPC A61K 8/9789; A61K 8/678; A61Q 5/12

(57) ABSTRACT

A composition containing mixture organic oils topically applied to prevent dry hair and hair loss as a pomade. Hair that is commonly lost through heat, chemicals, and styles, can benefit from all-natural hair and scalp moisturizer. Hair that has been subject to weave glues and extremely tight braids can also benefit from the lack of chemicals in product ingredients. The composition is safe to the human body, with no side effects thereto, with the composition of grapeseed oil, rosemary, vitamin E, cold pressed castor oil, shea butter, candelilla vegan wax, and lavender oil.

### BACKGROUND OF THE INVENTION

The current innovation relates to preparation for care of the hair, as a moisturizer applied to hair or scalp for protecting, grooming, adorning.

Hair grooming is important in keeping the general appearance of hair with strength, vitality, nourishment, dehydrated hair is free from moisture, evident in flaking, breakage and dry scalp.

As hair ingredient compositions increase, likewise the chemical compounds for extended shelf life and watered-down ingredients lead to drying the hair shaft, breakage, inferior product compilations.

Relaxer sales decline in hair perms as ethnicities move away from their use in choosing natural hair styles and hair moisturizing products without chemicals. There is a demand in the market for hair moisturizing products that are oil based that have been lab tested to grow mold and mildew and have a sustainable shelf life.

Alliance Analytical Laboratories, Inc. 179 West Randall Street, Coopersville, MI performed water activity test with result reading 0.69 molds have lower water activity limits there is no growth for any microbe with this score.

### BRIEF SUMMARY OF THE INVENTION

Hair moisturizer proportionately mixed includes grapeseed oil, fresh rosemary, cold press castor oil, lavender oil, vitamin E, organic shea butter, and candelilla vegan wax, the proceeding compilation is by approximation; Vitamin E one-twenty-fourth by weight, Lavender Oil one-twenty-fourth by weight, Cold Press Castor Oil one-twelfth by weight, Organic Shea Butter one-sixth by weight, Candelilla Wax one-sixth by weight, Fresh Rosemary one-third by weight, Grapeseed oil one-third by weight.

### BRIEF DESCRIPTION OF DRAWINGS

Drawings of the plant-based composition for castor beans, grape seeds, vitamin E, lavender, candelilla wax, rosemary, and shea butter. Omni Hair Moisturizer is a vegan product, FIGS. 1-7 are with summary, the compositions are unadapted, and farm grown.

FIG. 1 depicts a figured composition of the Castor Bean.

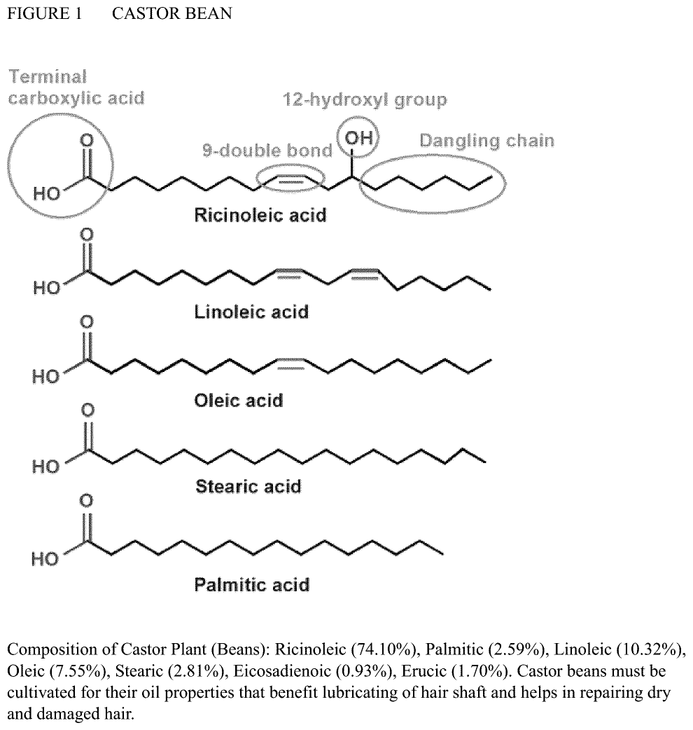

FIG. 2 depicts a figured composition of the Grape Seed.

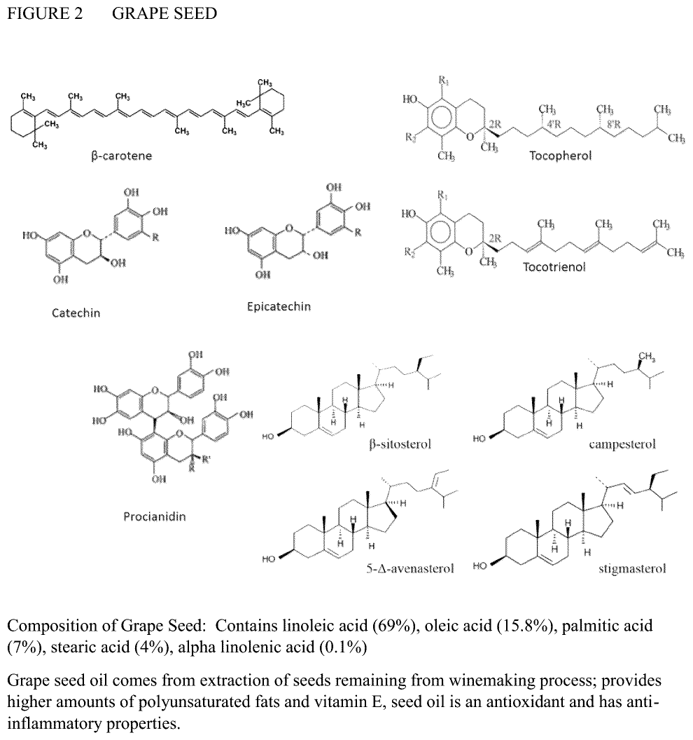

FIG. 3 depicts a figured composition of Vitamin E.

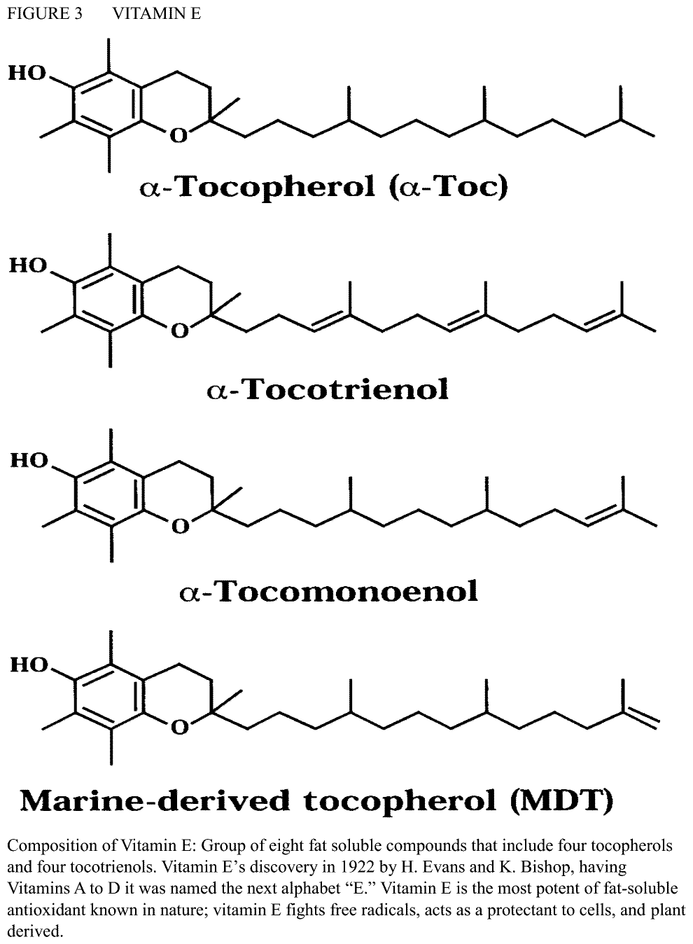

FIG. 4 depicts a figured composition of Lavender.

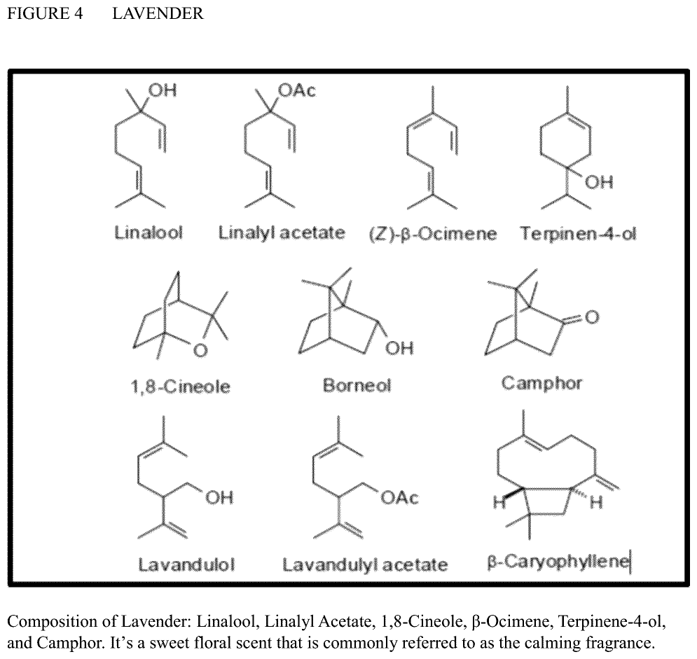

FIG. 5 depicts a figured composition of Candelilla Wax.

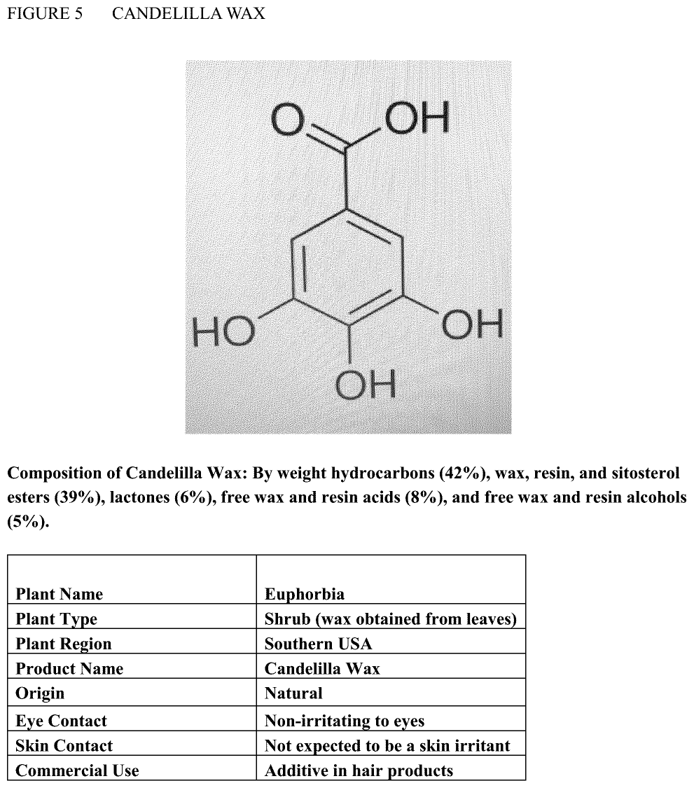

FIG. 6 depicts a figured composition of Rosemary.

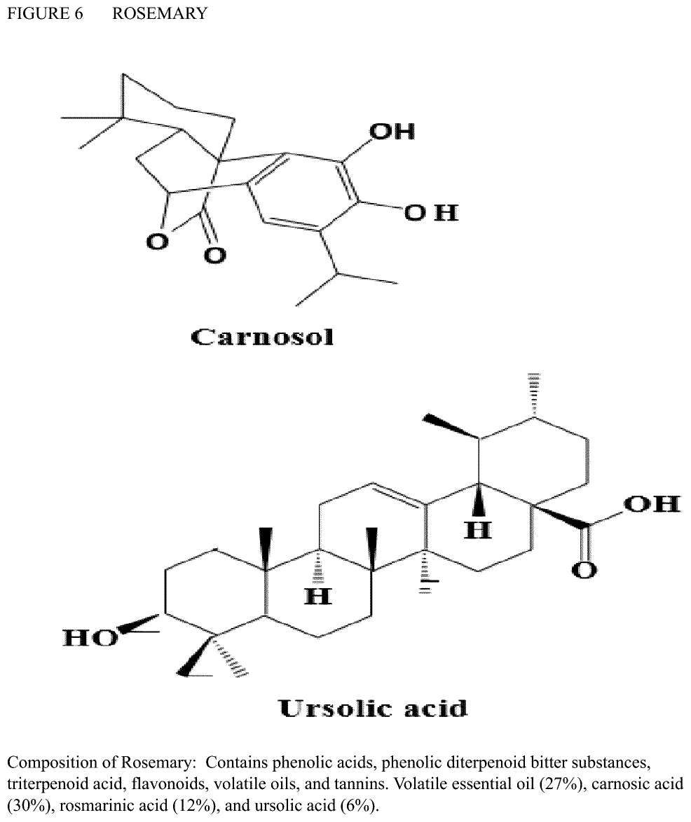

FIG. 7 depicts a figured composition of Shea Butter.

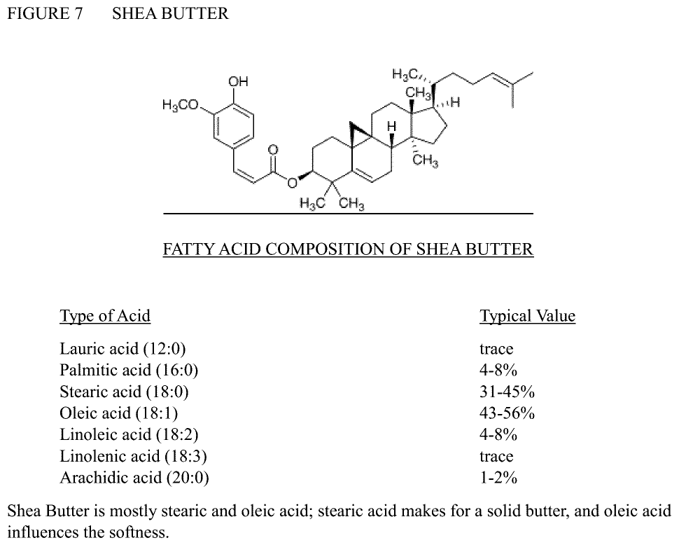

### DETAILED DESCRIPTION OF THE INVENTION

The general principles of hair moisturizer is to provide chemical free hair nourishment with approximate measures within a range of plus or minus five percent (5%), properly mixed together as follows: to make 12 ounces of the hair moisturizer the component parts may be approximately 4 oz of grapeseed oil 4 oz of fresh rosemary 2 oz of organic shea butter and cold press castor oil, 1 oz candelilla vegan wax, ½ oz vitamin E, and ½ oz lavender oil.

Hair moisturizer is applied with hands on post-washed wet hair apply to roots to create volume, for dry hair apply on the ends for texture. Specifically, the hydrating oils of shea butter, pure vitamin E, and cold pressed castor oil moisturize the skin.

These natural ingredients of present invention include:
1. Grapeseed oil
2. Fresh Rosemary
3. Vitamin E
4. Lavender Oil
5. Organic Shea Butter
6. Candelilla Wax
7. Cold Press Castor Oil
   Tools of this invention include:
1. Commercial 60 Quart Pot
2. Commercial Measuring Cups
3. Thermometer
4. Whisk
5. Micro Strainer
   Making 12 ounces of the hair moisturizer consists of the following in order:
approximately 4 oz of grapeseed oil
4 oz of fresh rosemary
2 oz of organic shea butter
cold press castor oil
1 oz candelilla vegan wax
½ oz vitamin E
½ oz lavender oil.
   When adding ingredients grapeseed oil is heated to 300 degrees F. (148 degrees C.), add fresh rosemary to oil and stir for four minutes, remove from heat and extract the rosemary, this is what gives hair moisturizer the natural green color without dyes and colorants, heat releases the rosemary oil. Approximately ten minutes of rest for oil and then add five remaining ingredients stirring to blend.

### Grapeseed Oil

Grapeseed oil can handle higher heat from styling tools, in comparison to olive oil, a popular ingredient in prior arts. Rosemary and castor oil fight inflammation. Grapeseed oil can withstand heats upward to 450 degrees.

### Fresh Rosemary

Rosemary has long been touted for its benefits, such as rejuvenating scalp and fighting dryness.

### Organic Shea Butter

Shea butter is derived from a nut and considered a fruit, full of fatty acids such as linoleic, oleic, stearic and palmitic acids that attribute to shine.

### Candelilla Vegan Wax

Candelilla Wax acts as barrier/sealant, prevents moisture loss in scalp/hair, and adds gloss.

### Cold Press Castor Oil

Cold Press Castor Oil brings balance to pH level of the scalp and its antiviral, antibacterial and antifungal. There are multiple benefits to castor oil than other ingredients, increases strength of hair, frizz control, protectant against breakage, and split ends.

### Lavender Oil

Lavender oil removes head lice and bacterial infections. This invention is not made with water as an additive, hence the composition of these seven ingredients prevents mold and mildew. Lavender oil is a mild and pleasing fragrance.

### Vitamin E

Vitamin E deeply moisturizes and protect hair from damage.

Omni Hair Moisturizer can be beneficial in a time when multiple ethnicities of women and men are gravitating to natural hair styles, and with this transition their looking for non-chemical hair products that promote moisture. People are savvy shoppers that stand in store isles reading labels. Invention consists of seven ingredients that work together to bring a natural plant-based product to consumers.

What is claimed is:

1. Omni Hair Moisturizer reduces dryness of the scalp, and works with curly, and natural hair type; comprising seven ingredients, as melted liquid or becoming a solid will not change composition; compared to prior arts the heat can ruin and spoil, others some heat leads to changing the product color and texture; but not with Omni Hair Moisturizer, the color is green with no artificial colorants or dyes; grapeseed oil can handle higher heat from styling tools, in comparison to olive oil a popular ingredient in other arts; rosemary and castor oil fight inflammation, lavender oil is a natural fragrance, vitamin E promotes healthy skin, organic shea butter relieves dryness, candelilla wax contributes to shine; Omni Hair Moisturizer was cosmetically tested and rated for water activity; scientific lab test score 0.69 for water activity, based on this score product less likely to grow mold and mildew.
